(19) United States
(12) Patent Application Publication
(10) Pub. No.: US 2025/0258122 A1
(43) Pub. Date: Aug. 14, 2025

(54) GAS SENSOR

(71) Applicant: TDK Corporation, Tokyo (JP)

(72) Inventors: Kei TANABE, Tokyo (JP); Sadaharu YONEDA, Tokyo (JP)

(21) Appl. No.: 19/196,874

(22) Filed: May 2, 2025

Related U.S. Application Data
(63) Continuation of application No. PCT/JP2022/043918, filed on Nov. 29, 2022.

(51) Int. Cl. G01N 27/14; G01N 33/00

(52) U.S. Cl. CPC G01N 27/14; G01N 33/0027

(57) ABSTRACT

Disclosed herein is a gas sensor that includes: first and second thermistors connected in series; first and second heaters configured to heat the first and second thermistors, respectively; and a control circuit configured to generate an output signal indicating a concentration of a gas to be measured based on a detection signal appearing at a connection point between the first and second thermistors in a state where the first and second heaters are heated. The control circuit is configured to control heating temperatures of the first and second heaters based on a resistance value of the first heater or second heater.

### BACKGROUND OF THE ART

### Field of the Art

The present disclosure relates to a gas sensor.

### Description of Related Art

Japanese Patent Publication JP 2021-089155A discloses a gas sensor configured to calculate the concentration of a gas to be measured based on the level of a detection signal appearing at the connection point between series-connected first and second thermistors. This gas sensor has a temperature sensor part including a third thermistor and controls the heating temperatures of the thermistors based on a temperature signal output from the temperature sensor part.

However, in the gas sensor described in JP 2021-089155A, a thermistor is used in the temperature sensor part, so that the number of required elements is disadvantageously increased. Further, the temperature coefficient of resistance of the thermistor is nonlinear, thus causing a measurement error in the temperature sensor part.

### SUMMARY

A gas sensor according to the present disclosure includes: first and second thermistors connected in series; first and second heaters configured to heat the first and second thermistors, respectively; and a control circuit configured to generate an output signal indicating a concentration of a gas to be measured based on a detection signal appearing at a connection point between the first and second thermistors in a state where the first and second heaters are heated. The control circuit is configured to control heating temperatures of the first and second heaters based on a resistance value of the first heater or second heater.

### BRIEF DESCRIPTION OF THE DRAWINGS

The above features and advantages of the present disclosure will be more apparent from the following description of some embodiments taken in conjunction with the accompanying drawings, in which:

FIG. 1 is a circuit diagram illustrating the configuration of a gas sensor 1 according to a first embodiment of the technology described herein;

FIG. 2 is a graph illustrating the relation between the temperature and resistance value of the heater resistors MH1 and MH2;

FIG. 3 is a graph illustrating the temperature characteristics of the thermistors Rd1 and Rd2;

FIG. 4 is a graph illustrating the relation between the temperature of the thermistors Rd1, Rd2 and their sensitivity to CO₂ gas;

FIG. 5 is a circuit diagram illustrating the configuration of a gas sensor 2 according to a second embodiment of the technology described herein;

FIG. 6 is a circuit diagram illustrating the configuration of a gas sensor 3 according to a third embodiment of the technology described herein; and

FIG. 7 is a circuit diagram illustrating the configuration of a gas sensor 4 according to a fourth embodiment of the technology described herein.

### DETAILED DESCRIPTION OF THE EMBODIMENTS

The present disclosure describes a gas sensor capable of accurately performing temperature measurement with a smaller number of elements and thereby of controlling the heating temperature of a heater more properly.

Some embodiments of the present disclosure will be explained below in detail with reference the accompanying drawings.

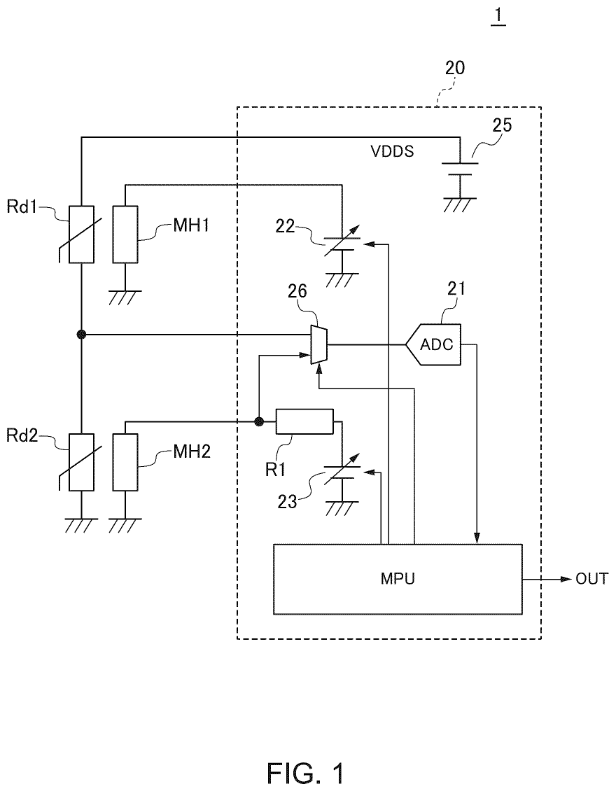

FIG. 1 is a circuit diagram illustrating the configuration of a gas sensor 1 according to a first embodiment of the technology described herein.

As illustrated in FIG. 1, the gas sensor 1 according to the first embodiment includes thermistors Rdl and Rd2, heater resistors MH1 and MH2 for heating the thermistors Rd1 and Rd2, respectively, and a control circuit 20 for controlling the heater resistors MH1 and MH2. Although not particularly limited, the gas sensor 1 according to the present embodiment is a thermal conduction type gas sensor for detecting the concentration of CO₂ gas in the atmosphere.

The thermistors Rd1 and Rd2 are detection elements made of a material having a negative temperature coefficient of resistance, such as a composite metal oxide, amorphous silicon, polysilicon, or germanium. Both the thermistors Rd1 and Rd2 detect the concentration of CO₂ gas, but have different operating temperatures, as described below. Here, the thermistor Rd1 constitutes a detection element, and the thermistor Rd2 constitutes a reference element. The thermistors Rd1 and Rd2 are connected in series between a power supply 25 for supplying a power supply potential VDDS and a ground, and a detection signal appearing at the connection point between the thermistors Rd1 and Rd2 is supplied to the control circuit 20.

The control circuit 20 includes an AD converter (ADC) 21, variable power supplies 22 and 23, an MPU 24, the power supply 25, a multiplexer 26, and a fixed resistor R1. The fixed resistor R1 is connected in series to the heater resistor MH2, and a temperature signal appearing at the connection point therebetween is supplied to the multiplexer 26. Under the control of the MPU 24, the multiplexer 26 supplies either the detection signal appearing at the connection point between the thermistors Rd1 and Rd2 or the temperature signal appearing at the connection point between the fixed resistor R1 and the heater resistor MH2 to the AD converter 21. The AD converter 21 AD-converts the detection signal or temperature signal and the resultant digital value to the MPU 24.

The MPU 24 selects the detection signal appearing at the connection point between the thermistors Rd1 and Rd2 during gas measurement operation, while it selects the temperature signal appearing at the connection point between the fixed resistor R1 and the heater resistor MH2 during resistance measurement operation. A plurality of the AD converters 21 may be used in place of the multiplexer 26. With this configuration, it is possible to execute the gas measurement operation and resistance measurement operation simultaneously. The MPU 24 controls the variable power supplies 22 and 23 based on the temperature signal obtained during the resistance measurement operation.

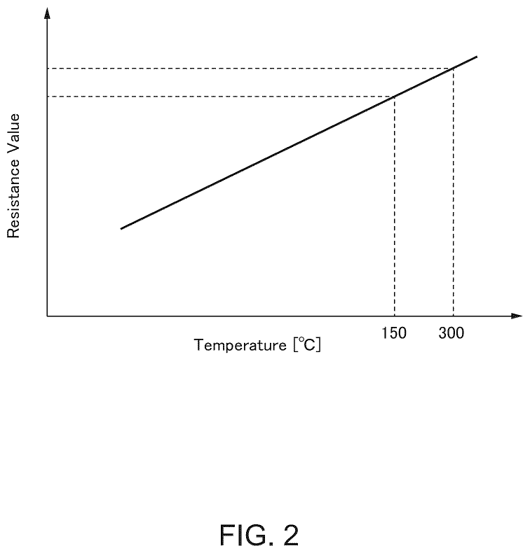

FIG. 2 is a graph illustrating the relation between the temperature and resistance value of the heater resistors MH1 and MH2. As can be seen from FIG. 2, the heater resistors MH1 and MH2 have a positive temperature coefficient of resistance that increases a resistance value as the temperature rises. In addition, unlike the thermistor, the relation between the temperature and the resistance value is linear. Examples of a conductive material having such characteristics include metal such as platinum. In the present embodiment, the fixed resistor R1 and heater resistor MH2 are connected in series. Thus, computation of the MPU 24 performed based on a temperature signal appearing at the connection point between the fixed resistor R1 and the heater resistor MH2 and an output voltage of the variable power supply 22 allows for measurement of the resistance value of the heater resistor MH2. Then, the resistance value of the heater resistor MH2 is converted to the temperature, whereby it is possible to accurately measure the current temperature of the heater resistor MH2. The heater resistor MH2 and thermistor Rd2 are disposed in close proximity to each other and thus, the temperature of the thermistor Rd2 can be regarded as substantially the same as the temperature of the heater resistor MH2. As described above, during the resistance measurement operation, it is possible to measure the temperature of the thermistor Rd2 by measuring the resistance value of the heater resistor MH2.

On the other hand, during the gas measurement operation, the heater resistors MH1 and MH2 are heated to 150° C. and 300° C., respectively, under the control of the MPU 24. The MPU 24 achieves this control by controlling output voltages output from the variable power supplies 22 and 23 based on the temperature signal obtained in the resistance measurement operation. That is, the MPU 24 controls the output voltage from the variable power supply 23 such that the temperature signal indicates that the heater resistor MH2 has a temperature of 300° C. On the other hand, the MPU 24 controls the output from the variable power supply 22 such that the heater resistor MH1 is heated to 150° C. by performing computation based on the characteristics illustrated in FIG. 2 with respect to the temperature signal.

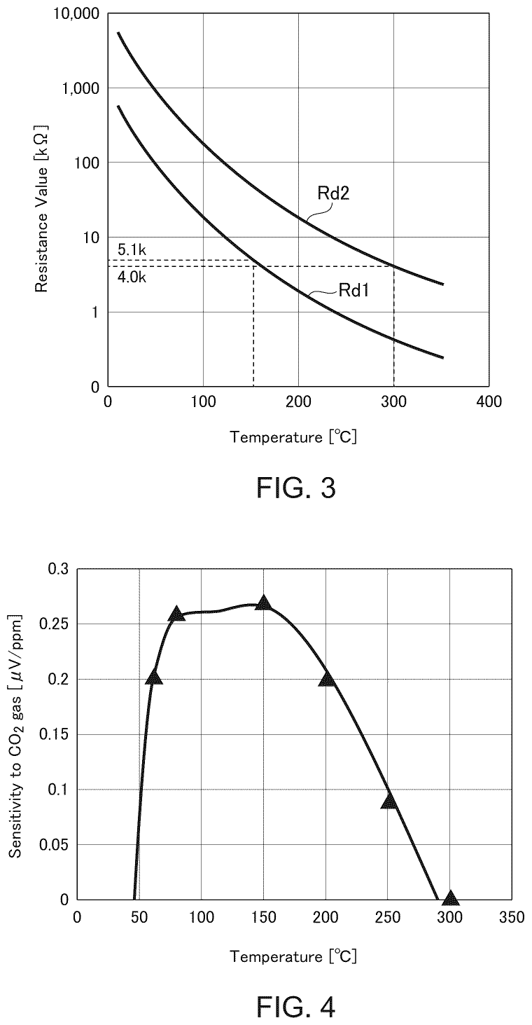

As illustrated in FIG. 3, the temperature characteristics of the thermistors Rd1 and Rd2 are mutually different and designed such that the resistance value of the thermistor Rd1 heated to 150° C. and the resistance value of the thermistor Rd2 heated to 300° C. are close to each other. In example illustrated in FIG. 3, the resistance value of the thermistor Rd1 heated to 150° C. is 5.1 kΩ, and the resistance value of the thermistor Rd2 heated to 300° C. is 4.0 kΩ. The resistance value of thermistor Rd1 heated to 150° C. and the resistance value of thermistor Rd2 heated to 300° C. may be approximately the same.

FIG. 4 is a graph illustrating the relation between the temperature of the thermistors Rd1, Rd2 and their sensitivity to CO₂ gas. As can be seen from the graph of

FIG. 4, the sensitivity of the thermistors Rd1 and Rd2 to CO₂ gas varies significantly depending on the temperature, and the sensitivity of the thermistors Rd1, Rd2 to CO₂ gas is almost zero in the temperature range below 40° C. or above 300° C. The sensitivity of the thermistors Rd1, Rd2 to CO₂ gas is maximum at about 150° C.

When CO₂ gas is present in the measurement atmosphere with the thermistor Rd1 as the detection element heated to 150° C., the heat dissipation characteristics of the thermistor Rd1 change according to the concentration. Such a change appears as a change in the resistance value of the thermistor Rd1. On the other hand, even when CO₂ gas is present in the measurement atmosphere with the thermistor Rd2 as the reference element heated to 300° C., the heat dissipation characteristics of the thermistor Rd2 hardly change according to the concentration. Therefore, the change in the resistance value of the thermistor Rd2 heated to 300° C. due to the concentration of CO₂ gas is sufficiently smaller than the change in the resistance value of the thermistor Rd1 heated to 150° C. due to the concentration of CO₂ gas. There is no problem if the resistance value of the thermistor Rd2 heated to 300° C. due to the concentration of CO₂ gas hardly changes.

As a result, the level of the detection signal appearing at the connection point between the thermistors Rd1 and Rd2 being heated changes according to the concentration of CO₂ gas in the measurement atmosphere. The detection signal is supplied to the MPU 24 through the AD converter 21, and the MPU 24 generates an output signal OUT indicating the concentration of CO₂ gas based on the supplied detection signal.

As described above, the gas sensor 1 according to the present embodiment measures the resistance value of the heater resistor MH2 without using a dedicated temperature sensor part to thereby calculate the temperature of the thermistor Rd2, thus making it possible to reduce the number of required elements. In addition, the material used for the heater resistors MH1 and MH2 has a linear temperature coefficient of resistance, reducing a measurement error, which allows for accurate temperature measurement. Further, in the present embodiment, since the resistance value of the heater resistor MH2 which is heated to a higher temperature is measured, a measurement error can be further reduced. In particular, the thermistor Rd1 heated to 150° C. undergoes a change in its heat dissipation characteristics based on the concentration of CO₂ gas in the atmosphere, so that the relation between the temperature of the heater resistor MH1 and the temperature of the thermistor Rd1 slightly changes due to the concentration of CO₂ gas; however, the thermistor Rd2 heated to 300° C. hardly undergoes a change in its heat dissipation characteristics based on the concentration of CO₂ gas in the atmosphere, so that the relation between the temperature of the heater resistor MH2 and the temperature of the thermistor Rd2 hardly changes due to the concentration of CO₂ gas. Thus, it is possible to perform temperature measurement more accurately by measuring the resistance value of the heater resistor MH2.

In the present embodiment, the resistance value of the heater resistor MH2 is measured, and the voltages output from the variable power supplies 22 and 23 are adjusted based on the measured resistance value; however, the resistance values of the heater resistors MH1 and MH2 may be respectively measured, and voltages output from the variable power supplies 22 and 23 may be adjusted based on the resistance values of the heater resistors MH1 and MH2, respectively.

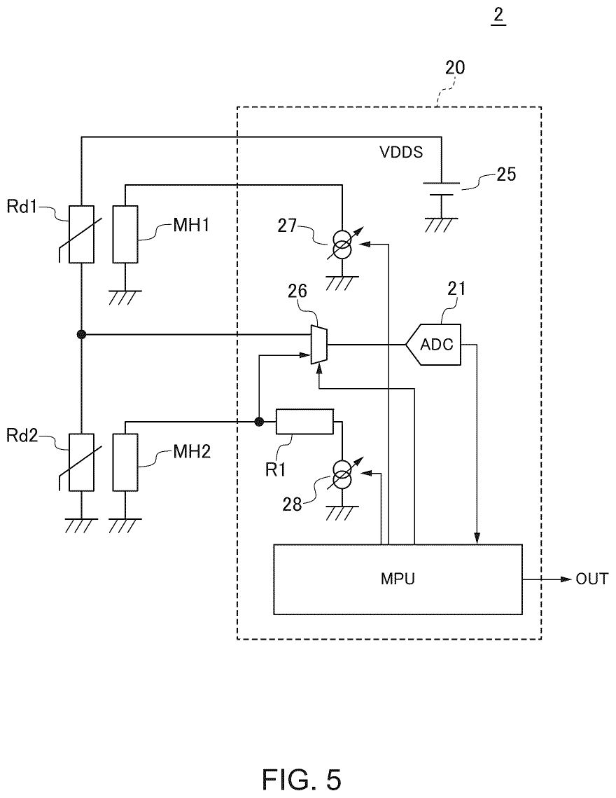

FIG. 5 is a circuit diagram illustrating the configuration of a gas sensor 2 according to a second embodiment of the technology described herein.

As illustrated in FIG. 5, the gas sensor 2 according to the second embodiment differs from the gas sensor 1 according to the first embodiment in that variable current sources 27 and 28 are used in place of the variable power supplies 22 and 23. With this configuration, an output current from the variable current source 27 is supplied to the heater MH1, and an output current from the variable current source 28 is supplied to the heater MH2. Other basic configurations are the same as those of the gas sensor 1 according to the first embodiment, so the same reference numerals are given to the same elements, and overlapping description will be omitted.

In the present embodiment, the amount of current to be supplied, based on the temperature signal, from the variable current source 27 (28) to the heater resistor MH1 (MH2) is controlled to heat the heater resistors MH1 and MH2 to 150° C. and 300° C., respectively. As exemplified in the present embodiment, the heating temperatures of the heater resistors MH1 and MH2 may be adjusted by current control. In this case, the fixed resistors R1 may be omitted.

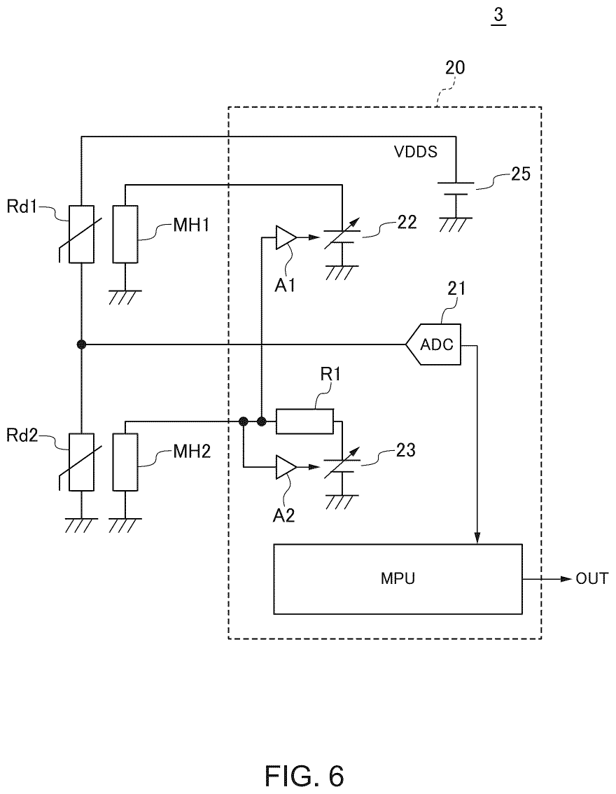

FIG. 6 is a circuit diagram illustrating the configuration of a gas sensor 3 according to a third embodiment of the technology described herein.

As illustrated in FIG. 6, the gas sensor 3 according to the third embodiment differs from the gas sensor 1 according to the first embodiment in that it has amplifiers A1 and A2 that receive the temperature signal and that the output voltages of the variable power supplies 22 and 23 are controlled based on the output signals of the amplifiers A1 and A2. Other basic configurations are the same as those of the gas sensor 1 according to the first embodiment, so the same reference numerals are given to the same elements, and overlapping description will be omitted.

The amplifiers A1 and A2 amplify the temperature signal to generate control signals and supply the generated control signals to the variable power supplies 22 and 23, respectively. The amplifiers A1 and A2 have mutually different gains according to their target heating temperatures. That is, the gain of the amplifier A1 for controlling the voltage value of the variable power supply 22 is set such that the heater resistor MH1 is heated to 150° C. according to a control signal generated based on the temperature signal, and the gain of the amplifier A2 for controlling the voltage value of the variable power supply 23 is set such that the heater resistor MH2 is heated to 300° C. according to a control signal generated based on the temperature signal.

As exemplified in the present embodiment, the variable power supplies 22 and 23 need not necessarily be subjected to digital control using the MPU 24 but may be subjected to analog control using the amplifiers A1 and A2. This reduces processing burden on the MPU 24 and prevents the occurrence of a temperature error due to the conversion error of the AD converter 21.

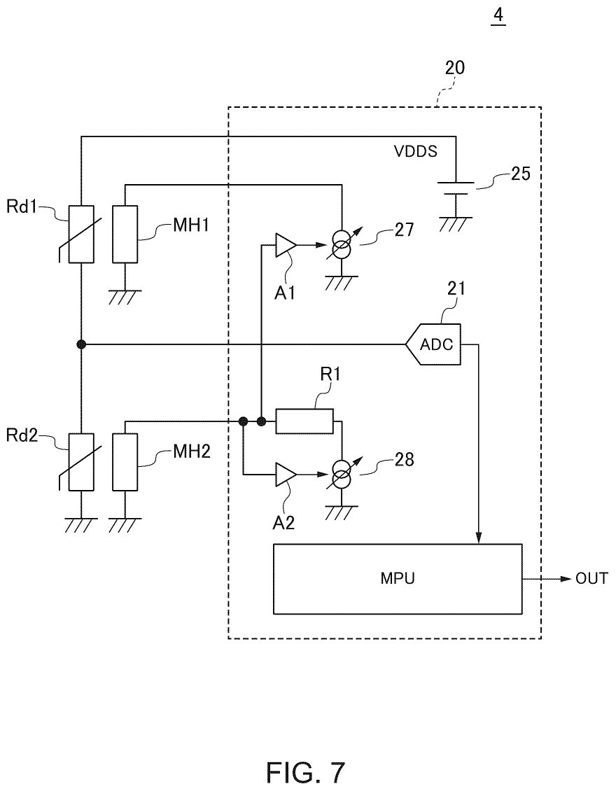

FIG. 7 is a circuit diagram illustrating the configuration of a gas sensor 4 according to a fourth embodiment of the technology described herein.

As illustrated in FIG. 7, the gas sensor 4 according to the fourth embodiment differs from the gas sensor 2 according to the second embodiment in that it has amplifiers A1 and A2 that receive the temperature signal and that the currents output from the variable current sources 27 and 28 are controlled based on the output signals of the amplifiers A1 and A2, respectively. Other basic configurations are the same as those of the gas sensor 2 according to the second embodiment, so the same reference numerals are given to the same elements, and overlapping description will be omitted.

The amplifiers A1 and A2 amplify the temperature signal to generate control signals and supply the generated control signals to the variable current sources 27 and 28, respectively. The amplifiers A1 and A2 have mutually different gains according to their target heating temperatures. That is, the gain of the amplifier A1 for controlling the current value of the variable current source 27 is set such that the heater resistor MH1 is heated to 150° C. according to a control signal generated based on the temperature signal, and the gain of the amplifier A2 for controlling the current value of the variable current source 28 is set such that the heater resistor MH2 is heated to 200° C. according to a control signal generated based on the temperature signal.

While some embodiments of the technology according to the present disclosure have been described, the technology according to the present disclosure is not limited to the above embodiments, and various modifications may be made within the scope of the present disclosure, and all such modifications are included in the technology according to the present disclosure.

For example, although the measurement target gas is CO₂ gas in the above embodiments, the present invention is not limited to this. Further, the sensor part used in the present invention need not necessarily be a thermal conduction type sensor, but may be a sensor of other types such as a catalytic combustion type. As an example, when the measurement target gas is CO gas, a catalytic combustion type sensor part can be used.

The technology according to the present disclosure includes the following configuration examples, but not limited thereto.

A gas sensor according to the present disclosure includes: first and second thermistors connected in series; first and second heaters configured to heat the first and second thermistors, respectively; and a control circuit configured to generate an output signal indicating a concentration of a gas to be measured based on a detection signal appearing at a connection point between the first and second thermistors in a state where the first and second heaters are heated. The control circuit is configured to control heating temperatures of the first and second heaters based on a resistance value of the first heater or second heater. With this configuration, it is possible to control the heating temperatures of the first and second heaters without using a dedicated temperature sensor part.

In the above gas sensor, the control circuit may be configured to generate the output signal based on the detection signal appearing at the connection point in a control state where the control circuit controls the heating temperatures of the first and second heaters based on the resistance value of one of the first and second heaters obtained in a state where the first and second heater are heated. This allows for the control of the heating temperatures of the first and second heaters without measuring the resistance value of the other one of the first and second heaters.

In the above gas sensor, the first and second heaters may be made of a conductive material having a positive temperature coefficient of resistance, the first heater is configured to be heated to a first temperature, the second heater is configured to be heated to a second temperature higher than the first temperature, and the control circuit is configured to control the heating temperatures of the first and second heaters based on a resistance value of the second heater obtained in a state where the second heater is heated. This allows for more accurate temperature measurement.

In the above gas sensor, the control circuit may include a first variable power supply configured to apply a voltage to the first heater and a second variable power supply configured to apply a voltage to the second heater and may be configured to control output voltages of the first and second variable power supplies based on the resistance value of the second heater obtained in a state where the second heater is heated. This allows the heating temperatures of the first and second heaters to be controlled by voltage control.

In the above gas sensor, the control circuit may include a first variable current source configured to supply a current to the first heater and a second variable current source configured to supply a current to the second heater and may be configured to control output currents of the first and second variable current sources based on the resistance value of the second heater obtained in a state where the second heater is heated. This allows the heating temperatures of the first and second heaters to be controlled by current control.

In the above gas sensor, the control circuit may include a fixed resistor connected in series to the second heater and may be configured to control the heating temperatures of the first and second heaters based on a temperature signal appearing at a connection point between the second heater and the fixed resistor. This facilitates measurement of the resistance value of the second heater.

In the above gas sensor, the control circuit may further include an AD converter configured to convert the temperature signal into a digital value and an MPU configured to compute voltage or current values to be applied to the first and second heaters based on the digital value. This allows the heating temperatures of the first and second heaters to be controlled by digital control.

In the above gas sensor, the control circuit may further include a first amplifier configured to generate a first control signal based on the temperature signal and a second amplifier configured to generate a second control signal based on the temperature signal, voltage or current value to be applied to the first heater may configured to be controlled by an output signal of the first amplifier, and voltage or current value to be applied to the second heater may configured to be controlled by an output signal of the second amplifier. This allows the heating temperatures of the first and second heaters to be controlled by analog control.

What is claimed is:

1. A gas sensor comprising:
first and second thermistors connected in series;
first and second heaters configured to heat the first and second thermistors, respectively; and
a control circuit configured to generate an output signal indicating a concentration of a gas to be measured based on a detection signal appearing at a connection point between the first and second thermistors in a state where the first and second heaters are heated,
wherein the control circuit is configured to control heating temperatures of the first and second heaters based on a resistance value of the first heater or second heater.

2. The gas sensor as claimed in claim 1, wherein the control circuit is configured to generate the output signal based on the detection signal appearing at the connection point in a control state where the control circuit controls the heating temperatures of the first and second heaters based on the resistance value of one of the first and second heaters obtained in a state where the first and second heater are heated.

3. The gas sensor as claimed in claim 2,
wherein the first and second heaters are made of a conductive material having a positive temperature coefficient of resistance,
wherein the first heater is configured to be heated to a first temperature,
wherein the second heater is configured to be heated to a second temperature higher than the first temperature, and
wherein the control circuit is configured to control the heating temperatures of the first and second heaters based on a resistance value of the second heater obtained in a state where the second heater is heated.

4. The gas sensor as claimed in claim 3,
wherein the control circuit includes a first variable power supply configured to apply a voltage to the first heater and a second variable power supply configured to apply a voltage to the second heater, and
wherein the control circuit is configured to control output voltages of the first and second variable power supplies based on the resistance value of the second heater obtained in a state where the second heater is heated.

5. The gas sensor as claimed in claim 3,
wherein the control circuit includes a first variable current source configured to supply a current to the first heater and a second variable current source configured to supply a current to the second heater, and
wherein the control circuit is configured to control output currents of the first and second variable current sources based on the resistance value of the second heater obtained in a state where the second heater is heated.

6. The gas sensor as claimed in claim 3,
wherein the control circuit includes a fixed resistor connected in series to the second heater, and
wherein the control circuit is configured to control the heating temperatures of the first and second heaters based on a temperature signal appearing at a connection point between the second heater and the fixed resistor.

7. The gas sensor as claimed in claim 4,
wherein the control circuit includes a fixed resistor connected in series to the second heater, and
wherein the control circuit is configured to control the heating temperatures of the first and second heaters based on a temperature signal appearing at a connection point between the second heater and the fixed resistor.

8. The gas sensor as claimed in claim 7, wherein the control circuit further includes an AD converter configured to convert the temperature signal into a digital value and an MPU configured to compute voltage values to be applied to the first and second heaters based on the digital value.

9. The gas sensor as claimed in claim 7,
wherein the control circuit further includes a first amplifier configured to generate a first control signal based on the temperature signal and a second amplifier configured to generate a second control signal based on the temperature signal,
wherein a voltage value to be applied to the first heater is configured to be controlled by an output signal of the first amplifier, and
wherein a voltage value to be applied to the second heater is configured to be controlled by an output signal of the second amplifier.

10. The gas sensor as claimed in claim 5,
wherein the control circuit includes a fixed resistor connected in series to the second heater, and
wherein the control circuit is configured to control the heating temperatures of the first and second heaters based on a temperature signal appearing at a connection point between the second heater and the fixed resistor.

11. The gas sensor as claimed in claim 10, wherein the control circuit further includes an AD converter configured to convert the temperature signal into a digital value and an MPU configured to compute current values to be supplied to the first and second heaters based on the digital value.

12. The gas sensor as claimed in claim 10,
wherein the control circuit further includes a first amplifier configured to generate a first control signal based on the temperature signal and a second amplifier configured to generate a second control signal based on the temperature signal,
wherein a current value to be supplied to the first heater is configured to be controlled by an output signal of the first amplifier, and
wherein a current value to be supplied to the second heater is configured to be controlled by an output signal of the second amplifier.
